(19) United States
(12) Patent Application Publication
(10) Pub. No.: US 2023/0017081 A1
(43) Pub. Date: Jan. 19, 2023

(54) BIOSENSOR FOR DETECTION OF SARS-COV-2 SPIKE GLYCOPROTEIN AND RELATED METHODS

(71) Applicant: CARBON HOLDINGS INTELLECTUAL PROPERTIES, LLC, Sheridan, WY (US)

(72) Inventor: Garrett W. Lindemann, Buffalo, WY (US)

(21) Appl. No.: 17/864,025

(22) Filed: Jul. 13, 2022

Related U.S. Application Data
(60) Provisional application No. 63/221,402, filed on Jul. 13, 2021.

(51) Int. Cl. G01N 33/543; G01N 33/569; G01N 21/65; G01N 27/02; G01N 27/414

(52) U.S. Cl. CPC G01N 33/54366; G01N 33/56983; G01N 21/65; G01N 27/021; G01N 27/4145; G01N 2333/165; G01N 2469/10

(57) ABSTRACT

An example biosensor includes a substrate, a graphene layer disposed on the substrate, and a binding site bonded to the graphene. The binding site includes an antibody configured to bind a SARS-CoV-2 spike glycoprotein.

### CROSS-REFERENCE TO RELATED APPLICATIONS

This application claims priority to U.S. Provisional Patent Application No. 63/221,402 filed on 13 Jul. 2021, the disclosure of which is incorporated herein, in its entirety, by this reference.

### FIELD

The described embodiments relate generally to biosensors, and more particularly to graphene-based biosensors configured to detect a SARS-CoV-2 spike glycoprotein.

### BACKGROUND

Biosensors may be used in life sciences, clinical diagnostics, environmental monitoring, and medical research for affinity-based sensing, such as hybridization between complementary single strand DNA in a microarray or affinity binding of a matched sensitive biological element-antigen pair. Biosensors may include a biological recognition element and a transducer that converts a recognition event into a measurable electronic signal. While graphene has desirable electrical properties that can allow for its use in converting a biological recognition event into a measurable electronic signal, it can be expensive to produce and difficult to effectively functionalize or incorporate into usable technologies.

Coronaviruses can be difficult to treat because they are not sufficiently characterized. The emergence of these newly identified viruses highlights the need for the development of novel antiviral strategies. Severe acute respiratory syndrome coronavirus 2 (SARS-CoV-2) is a newly emergent coronavirus which causes a severe acute respiratory disease, COVID-19. In addition, this virus uses its spike glycoprotein for binding to the ACE 2 receptor and internalization to a target cell causing pulmonary inflammation, clotting issues, and vascular disease. Thus, this spike protein represents an attractive target for biosensors to detect the virus as well as detect shedding of spike protein in vaccinated individuals and shedding of virus by individuals with a subclinical infection.

### SUMMARY

Embodiments disclosed herein relate to biosensors, and more particularly to graphene based biosensors configured to detect a SARS-CoV-2 spike glycoprotein and methods to detect the SARS-CoV-2 spike glycoprotein. In some embodiments, a biosensor can include a substrate, a graphene layer disposed on the substrate and a binding site bonded to the graphene layer. In some embodiments, the graphene layer can include graphene formed from coal. In other embodiments, the graphene can be formed from environmental carbon dioxide through sequestration. In some embodiments, the binding site can include an antibody configured to bind a SARS-CoV-2 spike glycoprotein.

In some embodiments, the biosensor can include a first subsensor and a second subsensor spaced apart from the first subsensor. The first subsensor can include a first binding site and the second subsensor can include a second binding site that is different from the first binding site. In some embodiments, the first subsensor can include an antibody configured to bind a SARS-CoV-2 spike protein of a first variant and the second subsensor can include an antibody configured to bind a SARS-CoV-2 spike protein of a second variant. The first variant is different from the second variant. In some embodiments, the first variant can include a SARS-CoV-2 spike protein from a natural infection and the second variant can include a SARS-CoV-2 spike protein from a vaccine.

In some embodiments, the biosensor can be configured to detect the SARS-CoV-2 spike protein in at least one of a saliva sample or a blood sample. In some embodiments, the biosensor can be configured to determine a quantity of spike glycoprotein. In some embodiments, the antibody is polyclonal. The antibody can include at least one of a single-chain variable fragment (scFV) or an antigen-binding fragment (Fab). In some embodiments, the antibody includes IML anti-11 DehydroThromboxane. In some embodiments, the biosensor includes a Raman spectrometer. The biosensor can include at least one of an impedance-based detector or a field effect transistor (FET). In some embodiments, the biosensor includes a multi-analyte array.

In some embodiments, a method to detect a spike glycoprotein can include forming a biosensor by depositing at least one graphene layer on a substrate, forming an electrical contact on the at least one graphene layer or the substrate, and attaching one or more antibodies to the at least one graphene layer. The at least one graphene layer can include one or more binding sites and the one or more antibodies can be configured to bind a target from a sample.

In some embodiments, the sample includes at least one of saliva or blood and the target includes a SARS-CoV-2 spike protein. In some embodiments, the method further includes performing Raman spectroscopy on the sample.

In some embodiments, the one or more antibodies is polyclonal. The one or more antibodies can include a recombinant antibody, in some embodiments. The one or more antibodies include at least one of a single-chain variable fragment (scFV) or an antigen-binding fragment (Fab). In some embodiments, the one or more antibodies can be multispecific.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a schematic cross-sectional view of a biosensor, according to an embodiment.

FIG. 2A shows a system configured to analyze a sample, according to an embodiment.

FIG. 2B shows Raman spectrographs of graphene derived from coal.

FIG. 3 is a top view of a biosensor that includes a plurality of subsensors, according to an embodiment.

FIG. 4 illustrates a method of detecting a spike glycoprotein, according to an embodiment.

### DETAILED DESCRIPTION

The present disclosure relates to a biosensor including one or more antibodies configured to bind a target from a sample. In some embodiments, the target includes a SARS-CoV-2 spike glycoprotein from a sample of saliva and/or blood. Current testing for virus or viral infections, such as those associated with COVID-19, requires two or more positive results for diagnosis of infection. Some instruments used for determining infection may be point-of-care (POC) devices, while others are large instruments residing in high volume commercial labs. Some POC instruments can provide results as quickly as about five (5) minutes to about a few hours after the sample has been obtained from a patient or subject. However, in most cases, the current instruments are not equally or fully deployed to all locations needed to test for the specific analyte, marker, or biomarker, such as locations for disease detection and/or triage locations. Conversely, samples that are obtained and sent to large commercial or hospital-based testing laboratories can take up to several days for results to be returned. Thus, leaving patients and healthcare professionals in limbo awaiting results. The disclosed systems and methods are generally directed to diagnosing and/or treating patients suffering from or at risk of developing a disease or condition associated with one or more viruses.

Disclosed herein are devices, methods, and systems useful in quickly and directly (or indirectly) detecting various analytes, markers, and biomarkers, for example detection of the causative agent of 2019 coronavirus disease (COVID-19) and/or conditions caused by COVID-19, which may be referred to as severe acute respiratory syndrome coronavirus 2 (SARS-CoV-2). In many embodiments, the disclosed methods, devices, and systems are useful in detecting the causative agent and/or fragments thereof (e.g. virus particles and analytes and biomarkers derived therefrom). The disclosed biosensor devices may be used at the POC to analyze samples from various sources, such as nasal swabs, saliva samples, blood samples, etc. Use of the disclosed biosensors may help to avoid the need to have patients and health care professionals to rely upon indirect measurements and analysis of a potentially infected patient via RT-PCR results or symptoms that may be associated with one or more other illnesses.

The proposed devices, systems, and methods are useful in various aspects of diagnosing, analyzing, and identifying one or more biomarkers associated with a virus. In some embodiments, the disclosed technology may be useful in treating and/or monitoring treatment of one or more diseases and conditions associated with viral infection. In some embodiments, the virus and viral infection may be a coronavirus, for example COVID 19, SARS or MERS.

The disclosed methods, devices, and systems may include production of one or more components from graphene, for example graphene from coal or sequestered carbon dioxide. In many cases, the disclosed technology may involve compositions and methods that improve functionalization of the graphene. Functionalization of graphene may include attaching one or more of an analyte, a capture probe, or combinations thereof. In some embodiments, a biosensor surface may be functionalized to recognize more than one specific probe, analyte, biomarker, etc. Thus, in many embodiments, the disclosed biosensor may detect and/or measure the presence of an infectious agent or antibodies thereto, such that the results may indicate an active or past infection.

The disclosed methods, devices, and systems provide for improved capture and/or detection of one or more markers/reagents/biomarkers associated with a disease or condition. In many embodiments, the disease or condition is the presence of a virus, microbe, fragment thereof, or associated infection, such as a viral infection. In most cases, the disclosed compositions, methods, devices, and systems may provide for enhanced sensitivity and enhanced specificity of detection, analysis, or diagnosis. In many cases, the sample, for example a sample derived from one or more of blood, mucus, saliva, nasal swab, etc. may be analyzed with the disclosed compositions, devices, methods, and systems in less than 60, 45, 30, or fewer minutes.

The disclosed compositions, methods, devices, and systems may provide for a c-graphene-containing biosensor that provides for increased sensitivity and specificity for the specific analyte, marker, virus, or viral particle being queried. The disclosed graphene-containing biosensors may be especially useful in an epidemic or pandemic, for example a viral pandemic such as COVID-19. In many cases, portable device, such as a smart device, smart phone, tablet, reader may be useful in controlling or monitoring the disclosed sensor, as well as gathering, storing, processing, analyzing, and/or displaying information from the sensor. In many embodiments, the smart device may be able to transmit the gathered test information to one or more central processing locations.

Definitions

The term “diagnosis,” “identification,” “analysis,” etc. may refer to an assessment of whether a subject or patient suffers from a disease or harbors an infective particle, or not. In some cases, the diagnosis may not be 100% correct, as to either the presence or absence, or origin of the disease or infection, or to its severity. The term, however, refers to a statistically significant portion thereof, which may be determined by those of skill in the art, such as healthcare personnel, statisticians, technicians, etc. A diagnosis may also include a prognosis for the tested patient or subject.

The term “analyte,” “marker,” and “biomarker” may be used interchangeably to refer to a biological molecule, or a fragment of a biological molecule, the change and/or the detection of which can be correlated with a particular diagnosis, condition, or state. Such biomarkers include, but are not limited to, viruses, viral particles, proteins, cytokines, hormones, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof {e.g., glycoproteins, ribonucleoproteins, lipoproteins). Exemplary biomarkers may include proteins, peptides, peptide fragments, nucleic acid sequences, derived from COVID-19 and other infectious agents, and/or antibodies directed thereto. Thus, biomarkers may indicate an active infection or a past infection.

The terms “receptor,” “capture probe,” and/or “capture molecule” may refer to one or more molecules or compounds that may interact with an analyte to form a co-molecule or complex, or recognition pair. In many embodiments, the receptor or capture probe is an antibody specific for an analyte, or an epitope on the analyte. In many embodiments, two or more capture probes may bind to a given analyte, at the same or at different epitopes. If the epitopes recognized by a capture probe are different on the analyte, a “sandwich assay” may be used. The capture probe or receptor can be various compounds and molecules, including, without limitation, natural or synthetic single stranded or double stranded nucleic acids, proteins, peptides, nucleopeptides, antibodies, antibody fragments.

The term “affinity” may refer to the interaction between two molecules, for example antigen-antibody, or analyte-capture probe, and their ability to form and maintain a complex. Binding affinity may be assayed with various methods and techniques, including, without limitation, MRI, surface plasmon resonance, fourier transform spectroscopy, Raman spectroscopy, fluorescence spectroscopy, circular dichroism, nuclear magnetic resonance, mass spectrometry, atomic force microscope, paramagnetic probes, dual polarization interferometry, multi-parametric surface Plasmon resonance, ligand binding assay and radio ligand. In many embodiments, a single molecule may have a high binding affinity for a specific target/antigen/analyte and a low binding affinity for a different target/antigen/analyte, which may be referred to as a non-specific binding target/antigen/analyte. Specificity may be determined by a difference in binding affinity of at least 10-, 20-, 50-, 100-, 1000-, 10,000, 100,000-fold, or more.

“Antibody” may refer to any form of immunoglobulin that exhibits the desired biological activity, such as binding to an analyte or ligand, and or competing with other receptors for the same, and therefore covers, but is not limited to monoclonal, polyclonal, and multispecific antibodies (e.g., bispecific antibodies), IgG, IgM, IgA, IgY, etc. Also included in the term antibody are fragments, including antigen-binding fragments and analogues of antibodies, typically, but not confined to molecules having at least a portion of the antigen binding or variable regions (e.g. one or more CDRs) of a parent antibody. Also included in the term antibodies are antibodies from livestock, aviary, herbivore, and carnivore animals. In most cases, a fragment may retain all or some of the binding specificity and affinity of the parental antibody, at least about 10% of the parental binding affinity, such as at least about 10% 20%, 50%, 70%, 80%, 90%, 95% or 100% or more of the parental antibody's binding affinity for the target. Non-limiting examples of antibody fragments include, but are not limited to, Fab, Fab′, F(ab′)2, and Fv fragments; diabodies; linear antibodies; single-chain antibody molecules, e.g., sc-Fv, unibodies, nanobodies, domain antibodies, and multispecific antibodies, as well as any combination thereof.

The term “biosensor” may refer to any device, composition, or compound that may interact with one or more of the biomarkers in a way that may be recognized, recorded, or measured. In some cases, the biosensor may include one or more detection devices to monitor an interaction with a biomarker. The detection may be direct or indirect through a read out. In some embodiments, detection may be visual, chemical, or electrical.

The term “virus” may be used to describe various viral structures, particles, as well as components thereof, such as proteins and/or nucleic acids. In some embodiments, the virus may be intact or not intact, such as denatured or not yet fully formed, for example, when a host cell is disrupted to expose viral parts within the cell.

The term “viral infection” may refer to a constellation of symptoms or characteristics that a patient or subject may present upon or shortly after being infected by a virus. Symptoms may include fever, muscle soreness, joint pains, headache, skin rash, swollen lymph glands, shortness of breath, mental fog, confusion, etc. Viral infection may be diagnosed by physical exam, or testing of one or more samples obtained from the patient.

The term “patient” may refer to a human or non-human subject who is being treated, monitored, tested, or the like, in many cases for the presence of a condition, disease, or disorder, such as possible infection, for example by a virus. The test may be performed at home, nursing home, a testing facility, hospital, bedside, triage center, etc. usually, by a healthcare professional.

The term “sample” may refer to any specimen or bio specimen obtained from a patient suspected or having or at risk of having, or developing a disease or condition. In many embodiments, the bio specimen is obtained from a bodily fluid of the patient, such as blood, saliva, mucus, nasal secretion, tear, sweat, feces, urine, etc. In some embodiments, the bio specimen may be a tissue and/or cells from the patient.

The term “healthcare provider” may refer to a physician, for example a primary care, emergency, intensive care, pulmonary, infectious disease physician and others, as well as employees, affiliates, colleagues, assistants thereof, such as nurses, therapists, administrators, pharmacy personnel, technicians, lab technicians, etc.

The term “treatment” may refer to any procedure, protocol, or method may aid in the prevention and/or amelioration of a disease, condition, or disorder referred to herein or its symptoms. Treatment may also refer to curing the disease or condition, and/or reestablishment of a healthy, pre-disease status or condition in the patient or subject with respect to the disease and/or its symptoms. Amelioration may refer to an improvement in one or more markers, symptoms, indicators, or signs of the disease or condition. For example, where the disease is a viral infection, treatment may help to reduce a fever or rash, and/or help to restore independent breathing or reduce labored breathing.

The present disclosure can include a handheld graphene biosensor device capable of directly detecting a biomarker associated with or derived from COVID-19. In one embodiment, the device may detect viral particles with high sensitivity and specificity, for example with an LOD of about 100 viral particles or less per milliliter of sample. In many embodiments, the test may be completed to a positive or negative read-out in under about 45 minutes from the collection of the sample. In many embodiments, the sample may be derived from either a blood, nasal swab, or saliva sample obtained from a patient being tested.

Antibody-antigen pair(s), specific for the biomarker to be tested, will be selected based on their binding affinities. In some cases, binding affinity may be determined from Enzyme-Linked Immunosorbent Assay (ELISA), or other suitable test. ELISAs are well known in the art and involve the use of solid-phase enzyme immunoassay (EIA) to detect the presence of a ligand in a sample (typically liquid). Antibodies are usually used that recognize a specific analyte be measured. ELISA. In some embodiments, the ELISA may be a type of “sandwich assay.” This approach may be useful in detecting the target analyte. For example, in this assay, one reagent may capture the analyte, and second reagent by also recognize the analyte bound to the first reagent—for example a first antibody bound to a surface, and a second antibody (that may recognize a second epitope on the analyte) may recognize the bound analyte, wherein the second antibody carries a reporter that may be detected by the biosensor and/or enhance the sensitivity or amplify the signal of the readout.

Binding affinity of various analyte-receptor combinations may vary. In some embodiments, the analyte-receptor may be an antibody-antigen combination. Affinity for these interaction/combinations may affect LOD, sensitivity, and specificity of the biosensor.

The graphene biosensors will be functionalized by decorating various surfaces with a receptor molecule, for example an antibody that is specific for the analyte. In some embodiments, the antibodies may be identified using a process that does not reduce the sensitivity of the graphene and does not interfere with the binding affinity of the chosen antibodies. In one embodiment, P bonding may be used to facilitate functionalization.

After the disclosed graphene biosensor is functionalized with the receptor molecule (e.g. a COVID-19 specific antibody) control samples may be tested. In these cases, samples may be obtained under mock sampling conditions, which may aid in determining the ability to directly or indirectly detect COVID-19 viral particles at various LOD. In many embodiments, the sensitivity and specificity of the interaction may be examined to maximize the ability to detect and/or measure analyte concentrations. In most embodiments, a collection to read-out time may be less than about an hour, for example about 45 minutes.

Control samples may include various recombinant antigens that are specific for the receptor being tested—such as COVID-19-specific antibodies. In some embodiments, “live” COVID-19 containing samples may be acquired and tested from various sources, such as from the CDC. These mock/control samples and challenges will be performed in various assays, such as Phosphate Buffered Saline, a Nasal Matrix, and a Salvia Matrix.

The disclosed device can be robust, rugged, easy to use, and portable. In some embodiments, the disclosed biosensor may be the size of a smart phone, such as an iPhone-sized device. Various healthcare professionals may use the disclosed device with little training, for example, physicians, nurses, technicians, public health officials, first responders, law enforcement officers, and border control officials. In many embodiments, the disclosed device is useful in directly detecting COVID-19 virus, particles, and fragments thereof in nasal swabs and salvia of patients suspected of being infected, infected, or at risk of being infected by COVID-19. The disclosed biosensor may also be useful in identifying patients that have recovered from infection, and therefore having few or no active viruses.

Results from the presently disclosed graphene biosensor, may be combined with results from one or more of a POC RT-PCR test, a serological lateral flow assay, and various medical criteria (fever, rash, cognitive impairment, cough, shortness of breath, diarrhea, etc.) may be useful in determining whether a patient has an active infection, is shedding virus, has recovered, or is presenting with some other respiratory infection.

This allows for people entering at border checkpoints to be screened quickly, as well as without the difficulties of a nasal swab through the interrogation of a salvia sample. Further, it provides clinic, emergency room, and doctors office staff a device to quickly and easily determine a patient's infection status (infected, shedding, or recovered). This device should assist in decreasing the transmission of COVID-19 between individuals by quickly identifying those that are infected and/or shedding when used in parallel with other POC tests.

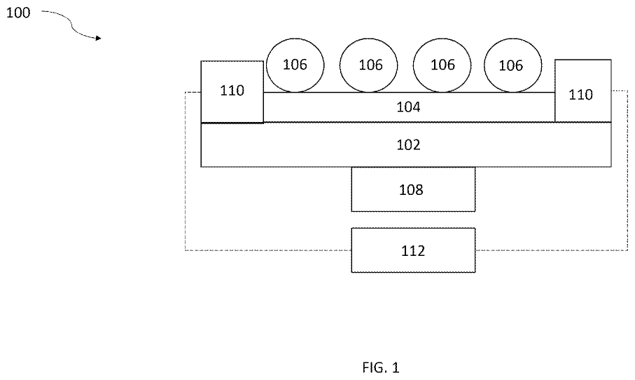

FIG. 1 is a schematic cross-sectional view of a biosensor 100. The biosensor 100 includes a substrate 102. The substrate 102 may include, for example, silica, silicon, a metal, or any other suitable material. In some embodiments, suitable substrates may include platinum, cobalt, nickel, copper, iron, iridium, gold, rubidium, rhenium, rhodium, germanium, and/or copper-nickel alloys. In other embodiments, suitable substrates may include silicon, silicon oxide, magnesium oxide, silicon dioxide, sapphire, h-BN, and/or silicon nitride. In yet other embodiments, bi-functional metals or trifunctional metals including copper germanium may be a suitable substrate. Copper and germanium may be included because of low solubility for carbon and an affinity for the formation of single layer graphene.

The substrate 102 may also include a single material (as shown) or may be formed from multiple layers (e.g., a base with at least one layer disposed thereon). At least one graphene layer 104 may be disposed on at least a portion of at least one surface of the substrate 102. In some examples, up to about 5 layers, 10 layers, 15 layers, 20 layers, can be disposed on the surface of the substrate 102.

In some examples, the graphene layer 104 can be synthesized by utilizing a metal catalyst. In some embodiments, transition metals (e.g. Platinum (Pt), Cobalt (Co), Nickel (Ni), Copper (Cu), Ruthenium (Ru), Iridium (Ir), Germanium (Ge)) may be included. Alloys, specifically Cu—Ni and Cu—Ge may also be used to make single and multi-layer graphene. In some examples, Silicon (Si) forms carbides due to the low diffusivity and high solubility of carbon on the surface of Si. Graphene may be synthesized direct onto everything from metal foils to insulators, and onto silicon chips with microelectronics for biosensors and sensors.

In some examples, the graphene may be exfoliated from a graphite present in coal to extract the graphene. Exfoliating the graphite includes separating the weakly bonded graphene layers of the graphite to extract the graphene, for example by electrical, chemical, and/or mechanical exfoliation. The graphite may be exfoliated using any suitable technique, such as at least one of liquid-phase exfoliation, electrochemical exfoliation, or micromechanical exfoliation.

Other techniques may extract graphene from coal. In some examples, graphene may be extracted from coal using coal or a coal-derived carbon source in a CVD process. In an embodiment, graphene may be extracted from coal using coal or a coal-derived carbon source in a flash Joule heating device. In some examples, any other suitable technique may extract graphene from coal.

In some examples, the graphene may be formed from environmental carbon dioxide (CO₂). In some embodiments, CO2 gas can be directly converted into multi-layer graphene via atmospheric pressure chemical vapor deposition (APCVD). Generally, the CO2 gas can be converted to methane, which can then be used to synthesize graphene by chemical vapor deposition. The first step includes the activation of CO₂ by passing a mixture of CO₂ and hydrogen gases through a metal (e.g. nickel) catalyst, leading to the production of methane. The second step includes the reduction of the methane or other reactive carbon species on a foil (e.g. copper) at high temperature, thus producing graphene. In other examples, a one-step synthesis of graphene using CO₂ gas, as carbon feedstock, and Cu—Pd substrates, employing an atmospheric pressure chemical vapor deposition (APCVD) reactor can be included. The Cu—Pd alloy acts not just as the catalyst for the CO₂ reduction and conversion, but also as the substrate for the graphene growth.

The graphene layer 104 may be disposed on the substrate 102 using any suitable method. For example, the graphene layer 104 may be disposed in a solution and the solution may be applied to the substrate 102 using a spin coating technique. One or more binding sites 106 configured to bind with or otherwise react with a target may be formed on the graphene layer 104. As previously discussed, the binding site 106 may be formed by at least one of functionalizing the graphene layer 104, attaching (i.e., directly or indirectly) one or more sensitive biological elements to the graphene layer 104, wrinkles or folds formed in the graphene layer 104, or impurities naturally present in the graphene layer 104. When the biosensor 100 includes a plurality of binding sites 106, each of the binding sites 106 may be the same or at least one of the binding sites 106 may differ from at least one other binding site 106.

In some embodiments, the graphene layer 104 can include a functionalized graphene to attach or bond at least one functionalization group to the graphene. When graphene is used in the biosensor 100, the functionalization groups may form binding site 106 of the biosensor 100 or the functionalization groups may form a portion of the binding site 106 (e.g., the binding site includes the functionalization group and an antibody). The binding site 106 of the biosensor 100 can be configured to bind or otherwise react with at least one target that is to be detected (e.g., an analyte, virus, antibody to the virus, bacteria, etc.). In some embodiments, the biosensor 100 can be configured to detect the SARS-CoV-2 spike glycoprotein in at least one of a saliva sample or a blood sample. Coronavirus enters into host cells by the attachment of transmembrane spike glycoprotein to the host surface receptor. Also, the spike (S) protein mediates the subsequent fusion between the viral and host cell membranes to make the entry of viral cell easier into the host cell. The S protein binds to ACE2, a transmembrane receptor that is widely expressed in the lungs, kidneys, gastrointestinal tissue, and heart. The binding to the host cell receptor and fusion between viral and cellular membranes have been performed with two subunits of the S protein: S1 subunit and S2 subunit, respectively. The S2 subunit contains neutralizing epitopes of the virus, including a conserved fusion peptide, a transmembrane domain, a cytoplasmic domain, and heptad repeats 1 and 2. In the S1 subunit, the core receptor binding domains are highly conserved. The differences in the amino acids are the cause of the direct interaction between spike protein and host cell receptor.

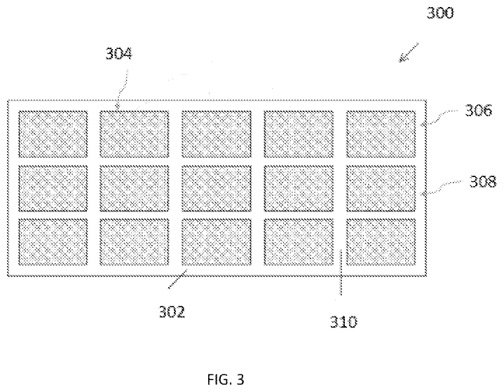

The functionalization groups may be added to the graphene layer 104 in any manner, as known in the art or as developed in the future. In some examples, only a single functionalization group is attached to the graphene. In such an example, the graphene may only detect one target or a plurality of undistinguishable targets. In some examples, a plurality of functionalization groups (e.g., about 2 to about 6, about 4 to about 8, about 6 to about 10, about 8 to about 15, about 10 to about 20, about 15 to about 30, about 25 to about 50, about 40 to about 70, or about 60 to about 100), such as different functionalization groups, may be added to the graphene, such as to the same graphene flake. In such examples, the graphene may detect a plurality of targets simultaneously. In some examples, the graphene is separated into a plurality of different groups of graphene that each include at least one flake of graphene. Each of the different groups of graphene may be functionalized with different functionalization groups. There may or may not be overlap between the different groups of graphene and the different functionalization groups. After functionalization, the different groups of graphene may detect different targets. The different graphene groups may form a plurality of subsensors on an array, wherein at least some of the subsensors are configured to detect different targets. A multi-analyte system, which allows detection of multiple biomarkers, is desirable for a quick, accurate, and early detection of the coronavirus or variants of the spike proteins. Multiplexing can be achieved by physically isolating different areas of the sensor surface, where each isolated area acts as a standalone sensor, describe below with reference to FIG. 3. Each of these individual areas could further be made specific to one type of biomarker and/or variant.

In some examples, the graphene formed during block 104 is not functionalized. The graphene may not be functionalized when the impurities, folds, or wrinkles in the graphene already form functionalization groups or binding sites for targets, when the sensitive biological element may be attached directly to the graphene, or when the biosensor is evaluated using techniques that do not require functionalization groups (e.g., Raman based detection detects the target based on the chemical structure of the targets).

In some examples, the binding site 106 may include an antibody attached or bonded to the graphene. The antibody may be attached to the graphene indirectly via the one or more functionalization groups or may be directly attached to the graphene. The antibody may be attached to the graphene using any suitable technique, such as exposing the antibody to the graphene while a stimulus is or is not applied to the graphene.

In some embodiments, the antibody is monoclonal. Monoclonal antibodies are laboratory-made proteins that mimic the immune system's ability to fight off harmful pathogens such as viruses, like SARS-CoV-2. In other embodiments, the antibody is polyclonal. Polyclonal antibodies are developed using several different immune cells. They will have the affinity for the same antigen but different epitopes, while monoclonal antibodies are developed using identical immune cells that are all clones of a specific parent cell. Like other infectious organisms, SARS-CoV-2 can mutate over time, resulting in genetic variation in the population of circulating viral strains. In some embodiments, the antibody includes at least one of a single-chain variable fragment (scFV) or an antigen-binding fragment (Fab). The antibody can include an IML anti-11 DehydroThromboxane.

The biosensor 100 may also include a heater 108 configured to heat at least the substrate 102 and the graphene layer 104. In an embodiment, the heater 108 may cause the target that is bound or otherwise reacted with the binding sites 106 to be released from the binding sites 106 by heating the graphene layer 104 allowing the biosensor 100 to be reused.

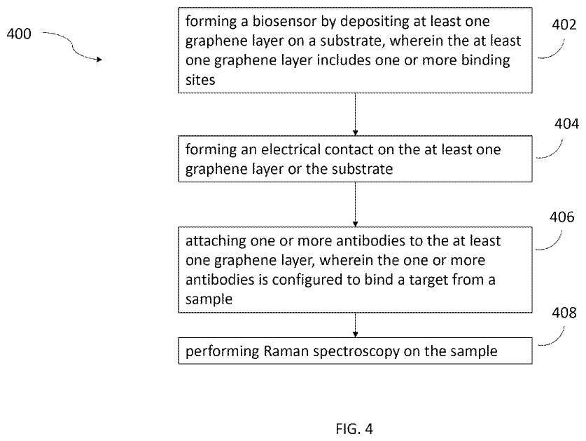

In some examples, the biosensor 100 includes two or more electrical contacts 110 (e.g., electrodes or probes) contacting at least a portion of the graphene layer 104. The electrical contacts 110 may also contact the substrate 102. The electrical contacts 110 may be connected to an electrical sensor 112 via one or more wires or other electrical connections. The electrical sensor 112 may include any sensor configured to detect one or more electrical characteristics of the graphene layer 104. For example, the electrical sensor 112 may include a voltmeter, a current sensor, a multimeter, or any other sensor that can detect the electrical characteristics of the graphene layer 104. For example, the electrical properties of the graphene layer 104 may change after the graphene layer 104 is exposed to the target (as shown in FIG. 4). How the electrical properties of the graphene layer 104 changes depends at least partially on the binding site 106 and the particular target. For example, the electrical current may change (i.e., the biosensor 100 is an amperometric biosensor), medium conductance may change (i.e., the biosensor 100 is a conductometric biosensor), the potential or charge accumulation may change (i.e., the biosensor 100 is a potentiometric biosensor), the interfacial electrical impedance may change (i.e., the biosensor 100 is a impedimetric sensor), or the current or potential across a semiconductor channel may change (i.e., the biosensor 100 is a field-effect transistor).

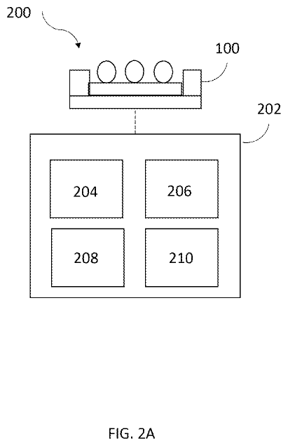

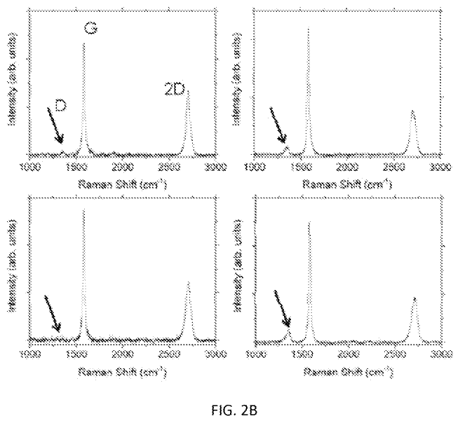

FIG. 2A shows a system configured to analyze a sample according to some embodiments. Biosensors are analytical devices that convert a biochemical/biological reaction into a measurable physio-chemical signal, which is proportional to the analyte concentration. The biosensor may include a rapid diagnostic biosensor, a sequencing biosensor, a cancer detection biosensor, a biosensor configured for personalized medicine, an enzyme-linked immunosorbent assay reporter, or any other biosensor. In some embodiments, the biosensor is configured to determine a quantity of spike glycoprotein. The biosensors disclosed herein may be more sensitive, specific robust, hardy, as well as potentially offering usage in more applications than existing biosensors while also being cheaper than biosensors that included graphene formed using conventional methods and sources, such as a sandwich assay.

The biosensor 100 may be part of a system 200 configured to detect a spike glycoprotein, in some embodiments. The system 200 can include electrical circuitry 202. In some examples, the electrical circuitry 202 is integrally formed with the biosensor 100. Regardless, the electrical circuitry 202 is configured to receive one or more signals from the biosensor 100. The signals from the biosensor 100 include the detected electrical properties of the graphene layer 104 and the electrical circuitry 202 is configured to analyze the detected electrical properties to determine if the target (e.g. a SARS-CoV-2 spike glycoprotein) is present. For example, the electrical circuitry 202 includes at least one processor 204 and non-transitory memory 206 coupled to the processor 204. The non-transitory memory 206 includes one or more operational instructions stored thereof and the processor 204 is configured to execute the operational instructions. The operational instructions, in conjunction with the signals received from the biosensor 100, allows the electrical circuitry 202 to determine the presence and/or quantity (e.g., concentration) of the SARS-CoV-2 spike glycoprotein or other target on the biosensor 100. For example, with the operational instructions, the processor 204 may determine the presence and/or quantity of the spike glycoprotein on the biosensor 100 by determining that the detected electrical properties include a current change, a medium conductance change, a potential or charge accumulation change, an interfacial electrical impedance change, or a current or potential across semiconductor channel.

The electric circuitry 202 may also include or be connected to an output 208 that allows the electrical circuitry 202 to communicate with an individual using the system 200. The output device 208 may include a display, one or more lights, a tactile feedback device, or any other suitable output device. The electrical circuitry 202 may be configured, through the output device 208, to provide graphic and/or tabular information to the individual, a yes or a no that the SARS-CoV-2 spike glycoprotein or other target is present or present over a certain quantity, the binding affinity (antibody/antigen) or mismatch of nucleic acids, the concentration of the target, or any other information. In some embodiments, the biosensor system 200 includes a Raman spectrometer, discussed in more detail in reference to FIG. 3.

In an embodiment, the electrical circuitry 202 may also include or be connected to an input 210 that allows a user to provide commands to the electrical circuitry 202, such as instructions to analyze a sample, which information to provide through the output 208, or information regarding the user. The input 210 may include a touch screen, a mouse, a keyboard, one or more buttons, or any other suitable input device.

The biosensor system 200 may include one or more components that are not shown. In some examples, the biosensor system 200 may include a housing that includes one or more components of the biosensor 100 or the system 200 disposed therein or thereon. The housing may be small enough to be easily held in a hand. In some examples, the biosensor 100 may include one or more stimulus devices (e.g., ultraviolet light source) that are configured to provide a stimulus that causes the target to be released from the binding sites. In some examples, the biosensor system 200 may include a power source, such as batteries or a plug that provides electrical power to one or more components (e.g., biosensor 100 and/or electrical circuitry 202) of the system 200.

In some embodiments, the biosensor system 200 can include at least one of an impedance-based detector or a field effect transistor (FET). Field-effect-transistors (FETs) are the key electronic biosensors widely applicable in the detection of biomolecules and pathogens. The main advantages of an electronic biosensor are miniaturization, low cost, and mass manufacturing. Besides, the concepts of a modular sensor for separating electrode and readout on a smartphone can be implemented effectively. FETs are easily fabricated and highly sensitive. They operate based on a semiconductor between a source and drain terminal, whose impedance is changed via the field effect of an applied electric field (via a gate terminal). When a molecule binds to the surface receptor, it changes the surface potential.

Referring back to FIG. 1, in some examples, one or more of the electrical contacts 110 or the electrical sensor 112 may be omitted from the biosensor 100. In some examples, the substrate 102, the graphene layer 104, the binding sites 106, and, optionally, the electrical contacts 110 may form a cartridge attached to the rest of the biosensor 100. The cartridge may decrease the cost of using the biosensor 100 since the electrical sensor 110 and the electrical circuitry 202 may be reused. The cartridge may also allow the biosensor 100 to detect multiple targets since the biosensor 100 may be configured to be used with different cartridges configured to detect different targets (e.g., the cartridges are interchangeable). Raman spectroscopy, microscopy, or other similar characterization techniques can then be used to analyze the biosensor 100 to determine if the SARS-CoV-2 spike glycoprotein is present on the biosensor 100.

FIG. 2B shows Raman spectrographs of graphene formed from coal according to the methods described herein. The Raman spectrographs shown in FIG. 2B were generated using samples of graphene formed by a CVD process using a coal-derived carbon source. The arrows in the graphs of FIG. 2B indicate the location of the “D band” of graphene. Generally, the “D band” of graphene in Raman spectrographs are associated with defect states in the graphene structure. The graphs of FIG. 2B illustrate that the “D band” is non-existent or minimal indicating that the graphene exhibits substantially no undesirable defects. The graphs illustrated in FIG. 2B may be baselines to determine if the biosensor 100 includes a desired level or impurities, the target molecule, or any other additional components. For example, Raman spectrographs that differ significantly from the graphs illustrated in FIG. 2B (e.g., including additional peaks and/or a change in the relative heights of the peaks) may indicate the presence of desired levels of impurities in the graphene as described herein. In some embodiments, Raman spectroscopy can indicate either the presence of a spike glycoprotein and/or a quantity of the spike glycoprotein in the sample. In some embodiments, the system 200 may provide an output and/or report that indicates the presence of a spike glycoprotein and/or a quantity of the spike glycoprotein in the sample.

In some examples, the biosensors disclosed herein may form multi-analyte array. FIG. 3 is a schematic top plan view of a biosensor 300 that is an array. An array may provide redundancy. In some embodiments, an array may provide an averaging of the results from the biosensors and/or control biosensors. The biosensor or biosensors forming the array can be the same or substantially similar to any of the biosensors disclosed herein. For example, the biosensor 300 includes a substrate 302. The biosensor 300 includes a plurality of subsensors 304 (shown textured) formed on the substrate 302. In some embodiments, the biosensor 300 includes a first subsensor 306 and a second subsensor 308. The first subsensor may include one or more first binding sites configured to bind or otherwise react with a first target while the second subsensor does not include the first binding sites and may not detect the first target. However, in some example, the second subsensor may include one or more second binding sites configured to bind or otherwise react with a second target that differs from the first target. The first subsensor may not include the one or more second binding sites and may not detect the second target.

The second subsensor 308 may be spaced apart from the first subsensor 306. In some embodiments, the first subsensor 306 can includes first binding site and the second subsensor 308 includes a second binding site that is different from the first binding site. In some embodiments, the first subsensor includes an antibody configured to bind a SARS-CoV-2 spike protein of a first variant and the second subsensor includes an antibody configured to bind a SARS-CoV-2 spike protein of a second variant. The second variant can be different from the first variant. In some embodiments, the first variant includes a SARS-CoV-2 spike protein from a natural infection and the second variant includes a SARS-CoV-2 spike protein from a vaccine. Further, in some embodiment, the first subsensor and/or second subsensor may include other antibodies configured to bind other proteins of the viral genomes. Viruses are small submicroscopic, obligate intracellular parasites, which contains either DNA or RNA as genome protected by a virus-encoded protein coat called capsid. The viral genome is packed inside a symmetric protein capsid, composed of either a single or multiple proteins; each of them is encoding a single viral gene. Viral genomes are considered as one of the most rapidly evolving organisms in biology due to their short replication time and a large amount of the offspring's released from the infected host cell. Viral proteins are grouped according to their functions, and groups of viral proteins include structural proteins, nonstructural proteins, regulatory proteins, and accessory proteins. Thus the first and/or second subsensor may include antibodies and/or other receptors (e.g. enzymes, nucleic acids, etc.) to detect an analyte.

Each of the subsensors 304 includes at least one graphene layer and one or more binding sites that are the same or substantially similar to any of the graphene layers or binding sites disclosed herein and may be formed according to any of the methods disclosed herein. Each of the subsensors 304 may be separated from each other by a gap 306. The gap 306 may be substantially free of graphene or substantially free of graphene that includes one or more binding sites. The gap 306 can be formed by masking a portion of the substrate 302 before depositing the graphene on the substrate 302, masking a portion of the graphene layer before the graphene layer is functionalized and/or attached to the antibody, the graphene layer is selectively formed around the gap 306, portions of the graphene layer are removed to form the gap 306, etc. When the biosensor 300 is configured to be analyzed using an electronic circuitry (not shown), the electric contacts may be configured to be connected to each of the subsensors 304, some of the subsensors 304, the gap 306, or a combination of the gap 306 and at least one of the subsensors 304.

The plurality of subsensors 304 may allow for the biosensor 300 to detect more targets than if the biosensor 300 only include one subsensor or a plurality of subsensors 304 that are all the same. Further, in some embodiments, the plurality of subsensors 304 may allow the biosensor 300 to determine a quantity of spike glycoprotein. At least some of the subsensors 304 may be different because, as previously discussed, the graphene used to form each subsensor 304 may be functionalized by different functionalization groups or have different antibodies attached thereto. Different sources of coal (e.g., coal from different regions, different mines, different seams, including different impurities) may also cause the graphene formed therefrom to bind or react with different targets or variants. In some examples, at least two of the subsensors 304 may be the same, which may allow the two subsensors 304 to confirm each other's results.

The biosensor 300 may include any suitable number of subsensors 304. For example, in the illustrated embodiment, the biosensor 300 includes 15 subsensors 304. However, it is noted that the biosensor 300 may include less than 15 subsensors 304 (e.g., 2, 6, 7, 10, 14, or ranges between any of these numbers) or greater than 15 subsensors 304 (e.g., 16 or greater, 25 or greater, 50 or greater, 75 or greater, 100 or greater, 16 to 30, 25 to 50, 40 to 70, or 60 to 100).

FIG. 4 illustrates a method 400 of detecting a spike glycoprotein, according to an embodiment. As illustrated in block 402, the method 400 includes forming a biosensor by depositing at least one graphene layer on a substrate, wherein the at least one graphene layer includes one or more binding sites. The graphene layer may be disposed on the substrate using any suitable method. The method 400 can further include forming an electrical contact on the at least one graphene layer or the substrate as shown at block 404. The electrical contact may be connected to an electrical sensor via one or more wires or other electrical connections in some embodiments. How the electrical properties of the graphene layer changes depends at least partially on the binding site and the particular target. The method 400 can further include, at block 406, attaching one or more antibodies to the at least one graphene layer. In some embodiments, the one or more antibodies is configured to bind a target from a sample. The target can include a SARS-CoV-2 spike protein. In some embodiments, the sample can include at least one of saliva or blood.

In some embodiments, the one or more antibodies is monoclonal. In some embodiments, the one or more antibodies includes a recombinant antibody. Recombinant antibodies (rAbs) are monoclonal antibodies, which are generated in vitro using synthetic genes. Unlike monoclonal antibodies (mAbs) which are produced using traditional hybridoma-based technologies, rAbs do not need hybridomas and animals in the production process. In some embodiments, the one or more antibodies include at least one of a single-chain variable fragment (scFV) or an antigen-binding fragment (Fab). A scFV is a fusion protein of the variable regions of the heavy (VH) and light chains (VL) of immunoglobulins, connected with a short linker peptide of ten to about 25 amino acids. This protein retains the specificity of the original immunoglobulin, despite removal of the constant regions and the introduction of the linker. The antigen-binding fragment (Fab) is a region on an antibody that binds to antigens. It is composed of one constant and one variable domain of each of the heavy and the light chain. The variable domain contains the paratope (the antigen-binding site), comprising a set of complementarity-determining regions, at the amino terminal end of the monomer. In other embodiments, the antibody is polyclonal. Polyclonal antibodies are developed using several different immune cells. They will have the affinity for the same antigen but different epitopes, while monoclonal antibodies are developed using identical immune cells that are all clones of a specific parent cell. In some embodiments, the one or more antibodies can be multispecific. The multispecific antibody combine two or more antigen-recognizing elements into a single molecule, able to bind to two or more targets.

In some embodiments, method 400 also includes performing Raman spectroscopy on the sample in block 408. As described above, Raman spectroscopy can identify the presence of a SARS-CoV-2 spike glycoprotein, the presence of variants, and/or a quantity of spike glycoprotein.

Various inventions have been described herein with reference to certain specific embodiments and examples. However, those skilled in the art recognize that many variations are possible without departing from the scope and spirit of the inventions disclosed herein, in that those inventions set forth in the claims below are intended to cover all variations and modifications of the inventions disclosed without departing from the spirit of the inventions. The terms “including” and “having” come as used in the specification and claims shall have the same meaning as the term “comprising.”

The foregoing description, for purposes of explanation, used specific nomenclature to provide a thorough understanding of the described embodiments. However, it can be apparent to one skilled in the art that the specific details are not required in order to practice the described embodiments. Thus, the foregoing descriptions of the specific embodiments described herein are presented for purposes of illustration and description. They are not target to be exhaustive or to limit the embodiments to the precise forms disclosed. It can be apparent to one of ordinary skill in the art that many modifications and variations are possible in view of the above teachings.

What is claimed is:

1. A biosensor, comprising:
a substrate;
a graphene layer disposed on the substrate; and
a binding site bonded to the graphene layer, the binding site including an antibody configured to bind a SARS-CoV-2 spike glycoprotein.

2. The biosensor of claim 1, further comprising:
a first subsensor; and
a second subsensor spaced apart from the first subsensor;
wherein the first subsensor includes a first binding site, and the second subsensor includes a second binding site that is different from the first binding site.

3. The biosensor of claim 2, wherein:
the first subsensor includes an antibody configured to bind a SARS-CoV-2 spike protein of a first variant;
the second subsensor includes an antibody configured to bind a SARS-CoV-2 spike protein of a second variant; and
the first variant is different from the second variant.

4. The biosensor of claim 3, wherein:
the first variant includes a SARS-CoV-2 spike protein from a natural infection; and
the second variant includes a SARS-CoV-2 spike protein from a vaccine.

5. The biosensor of claim 1, wherein the biosensor is configured to detect the SARS-CoV-2 spike protein in at least one of a saliva sample or a blood sample.

6. The biosensor of claim 1, wherein the biosensor is configured to determine a quantity of spike glycoprotein.

7. The biosensor of claim 1, wherein the antibody is polyclonal.

8. The biosensor of claim 1, wherein the antibody comprises at least one of a single-chain variable fragment (scFV) or an antigen-binding fragment (Fab).

9. The biosensor of claim 1, wherein the antibody comprises IML anti-11 DehydroThromboxane.

10. The biosensor of claim 1, wherein the biosensor comprises a Raman spectrometer.

11. The biosensor of claim 1, wherein the biosensor comprises at least one of an impedance-based detector or a field effect transistor (FET).

12. The biosensor of claim 1, wherein the biosensor comprises a multi-analyte array.

13. A method to detect a spike glycoprotein, comprising:
forming a biosensor by depositing at least one graphene layer on a substrate, wherein the at least one graphene layer includes one or more binding sites;
forming an electrical contact on the at least one graphene layer or the substrate; and
attaching an antibody to the at least one graphene layer, wherein the antibody is configured to bind a target from a sample.

14. The method of claim 13, wherein the target comprises a SARS-CoV-2 spike protein.

15. The method of claim 13, wherein the sample comprises at least one of saliva or blood.

16. The method of claim 13, further comprising performing Raman spectroscopy on the sample.

17. The method of claim 13, wherein the one or more antibodies is polyclonal.

18. The method of claim 13, wherein the one or more antibodies comprises a recombinant antibody.

19. The method of claim 13, wherein the one or more antibodies comprise at least one of a single-chain variable fragment (scFV) or an antigen-binding fragment (Fab).

20. The method of claim 13, wherein the one or more antibodies is multispecific.
